(19) United States
(12) Patent Application Publication
(10) Pub. No.: US 2006/0111434 A1
(43) Pub. Date: May 25, 2006

(54) Method for preparing derivatives of fungicidal iodochromones

(76) Inventors: Madeleine Delamare, Lyon (FR); Michel Casado, Saint Symphorien D'Ozon (FR)

Correspondence Address: OSTROLENK FABER GERB & SOFFEN, 1180 AVENUE OF THE AMERICAS, NEW YORK, NY, 100368403, US

(21) Appl. No.: 10/544,294

(22) PCT Filed: Jan. 28, 2004
(86) PCT No.: PCT/EP04/01786

§ 371 Date: Oct. 17, 2005

(30) Foreign Application Priority Data
Feb. 3, 2003 (EP) 03356014.5

(51) Int. Cl. A61K 31/353

(52) U.S. Cl. 514/456; 549/404

(57) ABSTRACT

This invention relates to new methods for preparing chemical compounds, in particular fungicidal compounds, especially iodinated derivatives of chromones, preferably compounds of formula (I), and intermediate compounds useful for these methods.

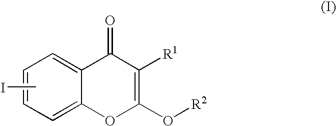

This invention relates to new methods for preparing chemical compounds, in particular fungicidal compounds, especially iodinated derivatives of chromones, preferably compounds of formula (I), and intermediate compounds useful for these methods.

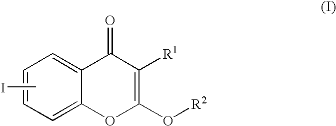

The subject of the present invention is also a method for preparing vinylalkoxides from vinylamines; it relates more particularly to the preparation of 2-halo and 2-alkoxychromones by the reaction of a 2-aminochromone with a copper halide or alkoxide.

The subject of the present invention is also a method for preparing 2-aminochromones by reacting an alkyl salicylate with an alkylnitrile and a base, followed by cyclization in an aqueous acid medium.

European patent application EP-861 242, which relates to fungicidal compounds derived from coumarin or chromone, generically reviews methods for preparing some of these compounds. However, even though two examples of iodinated compounds are disclosed, no specific method for preparing such iodinated derivatives is mentioned, nor is such an improved method suggested.

There are known from the prior art various methods allowing the preparation of aromatic halides from primary aromatic amines via a Sandmeyer reaction. There is known in particular the preparation of aromatic diazonium salts (March's Advanced Organic Chemistry, 5^{th} edition, John Wiley & Sons, Inc., (2001), p816-817 and 936) and the chemical reactions associated with such diazonium salts (Chem. Rev., (1988), 88, 765-792). However, none of the known methods describes such conversions to a vinyl ether series according to the scheme below in which * represents a free site, G¹ represents a halogen atom or an alkoxide and for which the structures are linear or cyclic which may be aromatic or otherwise:

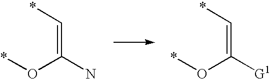

During the preparation of the fungal compounds covered by the present invention, the following problems are often encountered:
difficulty of improving the methods both from a technical point of view and from an economic point of view;
low yield;
insufficient purity;
difficulty of recycling the reagents used;
low rate of conversion;
reduction in the number of steps;
use of more economical agents;
improvement in the simplicity and safety of the reactions used;
limiting the number of by-products formed;
limiting side reactions.

The present invention makes it possible to solve all or some of these problems or disadvantages. It makes it possible in particular to avoid or to limit the substitution of halogen atoms by an alcoholate group when it is present, in particular when the structure comprises two halogen atoms.

A first aspect of the invention relates to the preparation of a compound of formula (I) 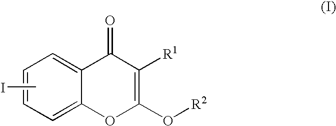
according to the following steps:
step A:
reaction of a compound of formula (V) 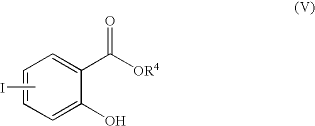
with a nitrile of formula R¹CH₂CN and a base to form the compound of formula (1); 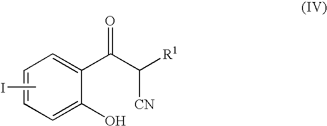
step B:
compound of formula (IV) which is then cyclized in an aqueous acid medium to form the compound of formula (III): 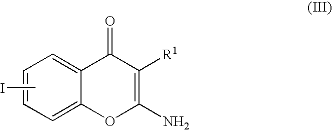
step C:
compound of formula (III) which gives the compound of formula (II) via a diazotization reaction by means of a compound chosen from sodium nitrite in the form of a mixture with an acid, alkyl nitrites, alkyl thionitrites and alkyl thionitrates, and decomposition by means of a compound AZₙ; 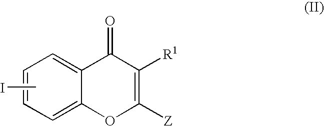
step D:
compound of formula (II) which gives the compound of formula (I) by the action of an alcohol in the form of a mixture with a base;
in the formulae (I) to (V)
R¹, R², R³ and R⁴, which are identical or different, represent a C₁-C₁₀ alkyl, C₁-C₁₀ alkenyl or C₁-C₁₀ alkynyl, one or more carbo- or heterocycles having 5 to 7 atoms, it being possible for these groups to be substituted or unsubstituted;
A represents a metal or a metal salt;
Z represents a group chosen from Cl, Br or —OR³;
n is equal to 0, 1 or 2.
   For R¹, noncyclic substituents or optionally aromatic cyclic substituents are preferred, and for Z, Cl is preferred.

When the groups or radicals of the compounds of formula (I) to (V) are substituted, they are preferably substituted with one or more groups which may be chosen, independently of each other, from an alkyl radical, alkenyl radical, alkynyl radical, a halogen atom, a cyano radical, trialkylsilyl radical, alkoxy radical, alkylthio radical, hydroxyl radical, nitro radical, amino radical, acyl radical, acyloxy radical, phenyl radical, heterocyclyl radical, phenylthio radical, phenoxy radical, heterocyclyloxy radical, heterocyclylthio radical and oxidized and optionally substituted derivatives of chemical entities containing a thio group.

The term heterocyclyl comprises heteroaryl groups and nonaromatic heterocyclyl groups, which may be saturated or unsaturated. The heteroaryl groups are generally 5- or 6-membered rings containing up to 4 heteroatoms chosen from nitrogen, oxygen and sulfur, optionally fused with a benzene ring. As examples of heteroaryl groups, there may be mentioned groups derived from thiophene, furan, pyrrole, thiazole, oxazole, imidazole, isothiazole, oxazole, pyrazole, 1,3,4-oxadiazole, 1,3,4-thiadiazole, 1,2,4-oxadiazole, 1,2,4-thiadiazole, 1,2,4-triazole, 1,2,3-triazole, tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, indazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine or pteridine.

The nonaromatic heterocyclyl groups are generally 3-, 5-, 6- or 7-membered rings containing up to 3 heteroatoms chosen from nitrogen, oxygen and sulfur, for example oxiranyl, thiiranyl, thiazolinyl, dioxolanyl, 1,3-benzoxazinyl, 1,3-benzothiazinyl, morpholino, pyrazolinyl, sulfolanyl, dihydroquinazolinyl, piperidinyl, phthalimido, tetrahydrofuranyl, tetrahydropyranyl, pyrrolidinyl, indolinyl, 2-oxopyrrolidino, 2-oxobenzoxazolin-3-yl or tetrahydroazepinyl.

The substituents, when they are present, on the phenyl or heterocyclyl groups may be for example halogen atoms, CN, NO₂, SF₅, B(OH)₂, trialkylsilyl, acyl, O-acyl or a radical E, OE or S(O)ₙE as defined above for R¹ or alternatively an optionally substituted amino radical; or alternatively two adjacent groups on the ring, together with the atoms to which they are attached form a carbocyclic or heterocyclic ring, which may be optionally substituted in a similar manner.

The term acyl comprises acid residues containing sulfur or phosphorus and carboxylic acid residues. Examples of acyl groups are thus —COR⁵, —COOR⁵, —CONR⁵R⁶, —CON(R⁵)OR⁶, —COONR⁵R⁶, —CON(R⁵)NR⁶R⁷, —COSR⁵, —CSSR⁵, —S(O)_{q}R⁵, —S(O)₂OR⁶, —S(O)_{q}NR⁵R⁶, —P(=L)(OR⁵)(OR⁶) or —COOR⁵, in which R⁵, R⁶ and R⁷, which may be identical or different, represent a hydrogen atom, an optionally substituted alkyl radical, optionally substituted cycloalkyl radical, optionally substituted cycloalkenyl radical, optionally substituted alkenyl radical, optionally substituted alkynyl radical, optionally substituted phenyl group or optionally substituted heterocyclyl group, or alternatively R⁵ and R⁶, or R⁶ and R⁷, together with the atom(s) to which they are attached, may form a ring, q represents 1 or 2 and L represents O or S.

Where appropriate, the amino radicals may be substituted for example with one or two optionally substituted alkyl or optionally substituted acyl radicals, or alternatively two substituents may form a ring, preferably a 5- to 7-membered ring, which may be substituted and which may contain other heteroatoms, such as for example morpholine.

Where appropriate, persons skilled in the art will know how to adapt the reaction conditions according to the groups present, in particular by suitably protecting them.

The combination of steps A and B, as well as the combination of steps C and D, taken separately constitute methods which also form part of the present invention as such. The reagents, the reaction conditions and the optional variant steps are similar to those described for the method of the invention comprising steps A to D.

The compounds of formula (V) may be prepared in a manner similar to what has been reported in the literature for alkyl 3-halosalicylates from equivalent alkyl salicylates, see for example Pharm. J., (1947), 159, 182.

During the condensation reaction of step A using the compound of formula (V) to give the compound of formula (IV), a homogeneous or heterogeneous inorganic or organic base, preferably a lithium alkylamide, for example lithium diisopropylamide, is advantageously used.

Advantageously, the base useful for this step may consist of a mixture of such bases. Persons skilled in the art will know how to determine the number and the relative quantities of these bases used as a mixture.

The compound of formula (IV) can be characterized but is generally not isolated.

According to a variant A′ of this step A, the nitrile of formula R¹CH₂CN may be replaced by a nitrile of formula R¹CXHCN in which X represents a halogen atom and the base is then replaced by a metal, in particular magnesium or zinc, see in particular Synth. Commun. (1989), 19(9-10), 1649-53.

During step B, the compound of formula (IV) gives the compound of formula (III). An inorganic or organic acid is used; ammonium chloride, hydrochloric acid or acetic acid are suitable. Aqueous acetic acid is preferred.

During step C, the compound of formula (III) gives the compound of formula (II) via a diazotization reaction, see for example March's Advanced Organic Chemistry, 5^{th} edition, John Wiley & Sons, Inc. (2001), p. 816, followed by a decomposition reaction.

As preferred reagents used for this diazotization reaction, sodium nitrite, and alkyl nitrites, in particular t-butyl or methyl nitrites, may be mentioned. During the use of NaNO₂, the preferred acids are HCl, HBr, H₂SO₄.

Preferably, the diazo intermediate may be decomposed by means of a compound AZₙ chosen from a metal halide or alkoxide, preferably CuCl, CuBr, CuCl₂, CuBr₂, CuOR³ or Cu(OR³)₂; CuCl₂ and CuBr₂ may also be used in their hydrated form.

The following combinations are particularly advantageous:
t-butyl or methyl nitrites and Cu(OR³)₂ or CUCl₂;
sodium nitrite with HCl or H₂SO₄ and CuOR³ or CuCl.

During this step C A therefore preferably represents copper. Other metals useful for this step are mentioned in Chem. Rev., (1988), 88, 765-792. This step C may also be carried out using NaNO₂ with ClSiMe₃ in order to prepare a compound in which Z represents Cl; persons skilled in the art will know how to adapt the conditions for such a Sandmeyer reaction known per se (Tet. Letters, 33 (22), 3167-3168 (1992)).

Likewise, NaNO₂, as a mixture with copper metal and KI may be used during this step C, in this case Z represents 1.

Likewise, HBF₄ as a mixture with NaNO₂ may be used. Z then represents F.

During step D, the compounds of formula (II) give the compound of formula (I). An alcohol of formula R²OH, in which R² represents a group such as those defined above, preferably a C₁-C₁₀ alkyl, still more preferably an n-butyl group, is used. The choice of the base used to form the alcoholate is within the capability of persons skilled in the art who may in particular use an alkali or alkaline-earth metal hydroxide or hydride, an alkali metal or an alkaline-earth metal, preferably KOH, NaH or sodium metal.

Another aspect of the present invention relates to a further improved method for preparing this same compound of formula (I) obtained directly by diazotization and decomposition of a compound of formula (III) by means of a compound AZₙ which is preferably a metal alkoxide. In this case, steps C and D of the method of the invention are replaced by a single step C′.

The various substituents of the compounds then have the same meanings as above.

Another variant of the method of the invention consists in replacing step D with step D′. In this case, a compound of formula (II) in which Z represents a group —OR³ is used, it is then possible to displace this group by a more appropriate similar group. For example, in the case where Z is an ethoxy group, it will be possible to displace it by a butoxy group which may be introduced by treating with sodium butoxide. The use of such a substitution constitutes an additional method of the present invention for the preparation of a compound of formula (I) starting from a compound of formula (II).

The methods of the invention which are particularly preferred allow the preparation of compounds of formula (I) by means of the compounds of formulae (II) to (V) in which the following characteristics are present alone or in combination:
the iodine atom is in the 6-position of the chromone;
R¹ represents a C₁-C₁₀ alkyl, preferably an n-propyl;
R² represents a C₁-C₁₀ alkyl, preferably an n-butyl;
R⁴ represents a C₁-C₁₀ alkyl, preferably a methyl;
A represents Cu;
Z represents a halogen atom, preferably Cl or Br, more preferably Cl, or the group —OR³ in which R³ represents a methyl or n-butyl group.

Most preferably, the methods of the invention are used for the preparation of a compound of formula (I) in which R¹ represents an n-propyl and R² represents an n-butyl.

As compounds which are advantageously prepared by means of the methods of the invention, there may be mentioned in particular
2-butoxy-6-iodo-3-propyl-4H-1-benzopyran-4-one
2-ethoxy-6-ioxo-3-propyl-4H-1-benzopyran-4-one
6-iodo-2-propoxy-3-propyl-4H-1-benzopyran-4-one
2-but-2-ynyloxy-6-iodo-3-propyl-4H-1-benzopyran-4-one
6-iodo-2-(1-methylbutoxy)-3-propyl-4H-1-benzopyran-4-one
2-but-3-enyloxy-6-iodo-3-propyl-4H-1-benzopyran-4-one
3-butyl-6-iodo-2-isopropoxy-4H-1-benzopyran-4-one
6-iodo-3-propyl-2-(tetrahydropyran-4-yloxy)-4H-1-benzopyran-4-one
6-iodo-3-propyl-2-(2,2,2-trifluoroethoxy)₄H-1-benzopyran-4-one

Moreover, persons skilled in the art will know how to adapt the methods of the invention to the preparation of the compounds of formula (I) to (V) in the form of their possible geometric and/or optical isomers, pure or as mixtures, in all proportions, including the possible racemic mixture(s), their optional N-oxides, addition salts with an acid, which are acceptable for use in the crop protection field, and their possible metal and/or metalloid complexes, which are acceptable for use in the crop protection field.

The methods of the invention have been described in relation to iodinated chromone derivatives. However, these methods may be adapted for the preparation of other halogenated chromone derivatives.

An additional aspect of the present invention relates to intermediate compounds useful for the methods of preparation according to the invention.

These compounds are the compounds of formula (III) and the compounds of formula (II) in which the various substituents have the same meaning as above.

Excluded however are the compounds of formula (II) for which the iodine atom is in the 6-position, R¹ represents an n-propyl group and R³ represents a methyl group or an n-propyl group.

Among the intermediate compounds of formula (III), there are preferred those in which the following characteristics are present alone or in combination:
the iodine atom is in the 6-position of the chromone;
R¹ represents a C₁-C₁₀ allyl group, preferably an n-propyl group.

Among the intermediate compounds of formula (II), there are preferred those in which the following characteristics are present alone or in combination:
the iodine atom is in the 6-position of the chromone;
R¹ represents a C₁-C₁₀ alkyl group;
Z represents a halogen atom.

More preferably, R¹ represents an n-propyl group and Z represents chlorine or bromine.

The following examples are given without limitation in order to further illustrate the present invention.

### EXAMPLE 1

### Preparation of 2-amino-6-iodo-3-propylchromone

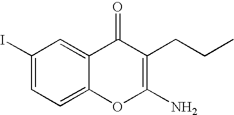

A solution of 8.4 ml (0.06 mol) of diisopropylamine in 20 ml of anhydrous THF, cooled to −20° C., is treated by adding 30 ml (0.06 mol) of 2 M n-butyllithium in solution in hexanes over 15 minutes. The solution is kept stirring at this temperature for 5 minutes and 2.10 ml (0.02 mol) of valeronitrile are added at −20° C. over 5 minutes. The solution is kept stirring at −20° C. for 15 minutes and a solution of 5.84 g (0.02 mol) of ethyl 4-iodosalicylate in 20 ml of anhydrous THF is added over 15 minutes. The suspension is heated to 5° C. and kept stirring for 45 minutes and then hydrolyzed with 15 ml of a saturated aqueous NH₄Cl solution. The biphasic solution is kept stirring for 2 hours. The aqueous phase is then removed, the organic phase is washed with 2×20 ml of water and then 20 ml of a saturated aqueous NaCl solution. The organic phase is dried over MgSO₄, filtered and concentrated to dryness, which gives 7.25 g of a yellow solid (70%). The product crystallized from acetonitrile has a melting point of 186-188° C.

### EXAMPLE 2

### Preparation of 2-chloro-6-iodo-3-propylchromone

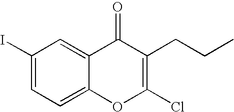

To a suspension of 1.98 g (6 mmol) of 2-amino-6-iodo-3-propylchromone, 1.22 g (7.2 mmol) of CuCl₂.2H₂O in 60 ml of acetonitrile, cooled to 0° C. is added 0.86 ml (7.2 mmol) of tert-butylnitrite over 10 minutes. The reaction mixture is heated to 20° C. and kept stirring at this temperature for 3 hours. The solution is hydrolyzed by adding 60 ml of water and extracted with 2×50 ml of ethyl acetate. The organic phases are combined and washed with 20 ml of NH₄OH, 2×50 ml of water and then 50 ml of a saturated aqueous NaCl solution. The organic phase is dried over MgSO₄, filtered and concentrated to dryness. This gives 2.04 g of a brown solid (78% theoretical yield) having a melting point of 103-105° C.

### EXAMPLE 3

### Preparation of 2-butoxy-6-iodo-3-propylchromone

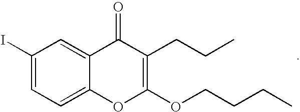

To a solution of 1.05 g (3 mmol) of 2-chloro-6-iodo-3-propylchromone in 10 ml of dichloromethane are added, at 20° C., 2.4 ml (4.3 mmol) of a 20% solution of sodium n-butoxide in n-butanol. The solution is kept at 20° C. for 2 hours and then heated at 40° C. for 4 hours. The reaction medium is hydrolyzed with 10 ml of water and the organic phase is washed with 2×10 ml of water, dried over MgSO₄, filtered and concentrated to dryness. This gives 0.95 g of a brown oil which crystallizes. The product crystallized from acetonitrile has a melting point of 69-71° C.

### EXAMPLE 4

### Preparation of 6-iodo-2-methoxy-3-propylchromone

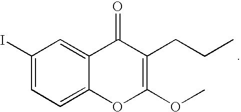

To a suspension of 0.31 g (0.95 mmol) of 2-amino-6-iodo-3-propylchromone, 0.15 g (1.2 mmol) of Cu(OMe)₂ in 2 ml of acetonitrile is added, at 20° C., 0.14 ml (1.2 mmol) of tert-butylnitrite over 5 minutes. The reaction medium is heated to 60° C. and kept stirring at this temperature for 3 hours. After returning to room temperature, the reaction mixture is hydrolyzed by adding 1 ml of water and extracted with 2×5 ml of ethyl acetate. The organic phases are combined and washed with 5 ml of NH₄OH, 2×5 ml of water and then 5 ml of a saturated aqueous NaCl solution. The organic phase is dried over MgSO₄, filtered and concentrated to dryness.

### EXAMPLE 5

### Preparation of 2-n-butoxy-6-iodo-3-propylchromone

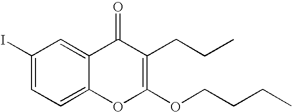

To a solution of 0.27 g (0.76 mol) of 2-ethoxy-6-iodo-3-propylchromone in 2.5 ml of n-butanol is added, at 20° C., 0.5 ml (0.91 mmol) of a 20% (w/w) solution of sodium n-butoxide in n-butanol over 5 minutes. The reaction medium is kept stirring at 20° C. for 2 hours. The reaction mixture is treated by adding 2 ml of an aqueous 0.1 N HCl solution and extracted with 10 ml of dichloromethane. The organic phase is extracted and washed with 2×5 ml of water. The organic phase is dried over MgSO₄, filtered and concentrated to dryness. This gives 0.24 g of a brown oil which crystallizes.

1. A method for preparing a compound of formula (I) 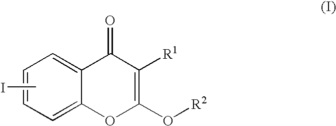
according to the following steps:
step A:
reaction of a compound of formula (V) 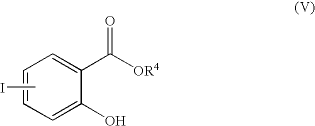
with a nitrile of formula R¹CH₂CN and a base to form the compound of formula (IV); 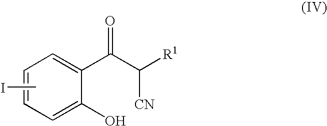
step B:
compound of formula (IV) which is then cyclized in an aqueous acid medium to form the compound of formula (III): 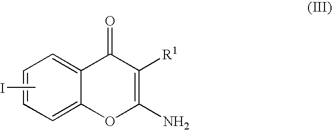
step C:
compound of formula (III) which gives the compound of formula (II) via a diazotization reaction by means of a compound chosen from sodium nitrite in the form of a mixture with an acid, alkyl nitrites, alkyl thionitrites and alkyl thionitrates, and decomposition by means of a compound AZₙ; 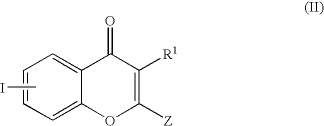
step D:
compound of formula (II) which gives the compound of formula (I) by the action of an alcohol in the form of a mixture with a base;
in the formulae (I) to (V)
R¹, R², R³ and R⁴, which are identical or different, represent a C₁-C₁₀ alkyl, C₁-C₁₀ alkenyl or C₁-C₁₀ alkynyl, one or more carbo- or heterocycles having 5 to 7 atoms, it being possible for these groups to be substituted or unsubstituted;
A represents a metal or a metal salt;
Z represents a group chosen from Cl, Br or —OR³;
n is equal to 0, 1 or 2.

2. A method according to claim 1, for which the base used during step A is lithium diisopropylamide.

3. A method according to claim 1, for which the acid used during step B is acetic acid.

4. A method according to claim 1, for which A is copper.

5. A method according to claim 1, in which step C uses t-butyl or methyl nitrite.

6. A method according to claim 1, for which AZₙ represents Cu(OR³)₂ or CuCl₂.

7. A method according to claim 1, in which step C uses t-butyl or methyl nitrite and for which AZₙ represents Cu(OR³)₂ or CuCl₂.

8. A method according to claim 1, in which step C uses sodium nitrite in the form of a mixture with HCl or H₂SO₄.

9. A method according to claim 1, for which AZₙ represents Cu(OR³) or CuCl.

10. A method according to claim 1, in which step C uses sodium nitrite in the form of a mixture with HCl or H₂SO₄ and for which AZₙ represents Cu(OR³) or CuCl.

11. A method according to claim 1, in which step D uses an alcohol of formula R²OH in which R² is a C₁-C₁₀ alkyl.

12. A method according to claim 11, for which R² represents n-butyl.

13. A method according to claim 1, in which step D is replaced by step D′ and in which there is used a compound of formula (II) in which Z represents a group —OR³, which is displaced by a similar, more appropriate group.

14. A method according to claim 13, for which Z is the ethoxy group which is displaced by a butoxy group introduced by treating with sodium butoxide.

15. A method according to claim 1, in which step A is replaced by step A′ and in which there is used a nitrite of formula R¹CXHCN, in which X represents a halogen atom, with, as base, a metal chosen from magnesium or zinc.

16. A method according to claim 1, for which the following characteristics are present alone or in combination:
the iodine atom is in the 6-position of the chromone;
R¹ represents a C₁-C₁₀ alkyl, preferably an n-propyl;
R² represents a C₁-C₁₀ alkyl, preferably an n-butyl;
R⁴ represents a C₁-C₁₀ alkyl, preferably a methyl;
A represents Cu;
Z represents Cl or Br, or the group —OR³ in which R³ represents a methyl or n-butyl group.

17. The method as claimed in claim 1, for which the preparation of a compound of formula (I) in which R¹ represents an n-propyl and R² represents an n-butyl.

18. A method for preparing a compound of formula (III) 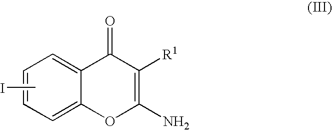
according to the following steps:
reaction of a compound of formula (V) 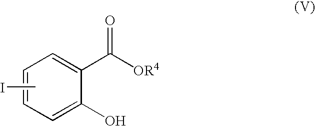
with a nitrile of formula R¹CH₂CN and a base to form the compound of formula (IV); 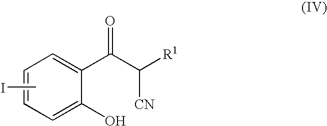
which compound of formula (IV) is then cyclized in an aqueous acid medium to form the compound of formula (III);
in formulae (III) to (V)
R¹, R², R³ and R⁴, which are identical or different, represent a C₁-C₁₀ alkyl, C₁-C₁₀ alkenyl or C₁-C₁₀ alkynyl, one or more carbo-heterocycles having from 5 to 7 atoms, it being possible for these groups to be substituted or unsubstituted.

19. A method for preparing a compound of formula (II) 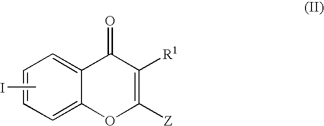
from a compound of formula (III) which gives the compound of formula (II) via a diazotization reaction 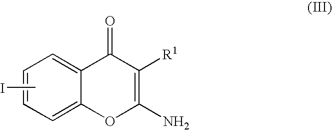
by means of a compound chosen from sodium nitrite in the form of a mixture with an acid, alkyl nitrites, alkyl thionitrites and alkyl thionitrates; and decomposition by means of a compound AZₙ;
in the formulae (II) and (III)
R¹ and R³, which are identical or different, represent a C₁-C₁₀ alkyl, C₁-C₁₀ alkenyl or C₁-C₁₀ alkynyl, one or more carbo- or heterocycles having 5 to 7 atoms, it being possible for these groups to be substituted or unsubstituted;
A represents a metal or a metal salt;
Z represents a group chosen from Cl, Br or —OR³;
n is equal to 0, 1 or 2.

20. A method for preparing a compound of formula (I) 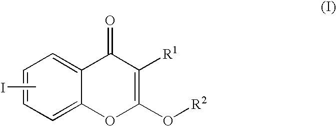
from a compound of formula (II) which gives the compound of formula (I) 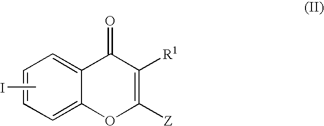
by the action of an alcohol in the form of a mixture with a base;
in the formulae (I) and (II)
R¹ and R², which are identical or different, represent a C₁-C₁₀ alkyl, C₁-C₁₀ alkenyl or C₁-C₁₀ alkynyl, one or more carbo- or heterocycles having 5 to 7 atoms, it being possible for these groups to be substituted or unsubstituted;
Z represents a group chosen from Cl, Br or —OR³.

21. A compound of formula (III) 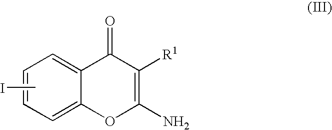
in which R¹ represents a C₁-C₁₀ alkyl, C₁-C₁₀ alkenyl or C₁-C₁₀ alkynyl, one or more carbo- or heterocycles having from 5 to 7 atoms, it being possible for these groups to be substituted or unsubstituted.

22. The compound as claimed in claim 21, of formula (III) in which the following characteristics are present alone or in combination:
the iodine atom is in the 6-position of the chromone;
R¹ represents a C₁-C₁₀ alkyl group.

23. The compound as claimed in claim 21, of formula (III) in which R¹ represents the n-propyl group.

24. A compound of formula (II) 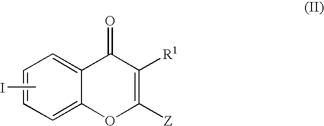
in which
R¹ and R³, which are identical or different, represent a C₁-C₁₀ alkyl, C₁-C₁₀ alkenyl or C₁-C₁₀ alkynyl, one or more carbo- or heterocycles having 5 to 7 atoms, it being possible for these groups to be substituted or unsubstituted;
Z represents a halogen atom.

25. A compound according to claim 24, of formula (II) in which the following characteristics are present alone or in combination:
the iodine atom is in the 6-position of the chromone;
R¹ represents a C₁-C₁₀ alkyl group.

26. A compound according to claim 24, of formula (II) in which R¹ represents an n-propyl group and Z represents chlorine or bromine.

27. A compound according to claim 25, of formula (II) in which R¹ represents an n-propyl group and Z represents chlorine or bromine.
